(19) United States
(12) Patent Application Publication
(10) Pub. No.: US 2012/0172856 A1
(43) Pub. Date: Jul. 5, 2012

(54) Laparoscopic Laser Device and Method

(75) Inventor: Kester Nahen, Heidelberg (DE)

(73) Assignee: AMS Research Corporation, Minnetonka, MN (US)

(21) Appl. No.: 13/418,247

(22) Filed: Mar. 12, 2012

Related U.S. Application Data
(62) Division of application No. 11/671,071, filed on Feb. 5, 2007.
(60) Provisional application No. 60/765,879, filed on Feb. 7, 2006.

(51) Int. Cl. A61B 18/20

(52) U.S. Cl. 606/14

(57) ABSTRACT

Laser radiation delivered to a treatment area may be used with a smoke suppressing irrigant. A laparoscopic laser device may include an elongate body adapted for insertion into an insufflated bodily cavity. A laser energy delivery element, at the distal end of the elongate body, may be coupleable to a source of tissue-vaporization-capable laser energy and capable of delivering laser energy along a laser energy path extending away from the laser energy delivery element. A smoke-suppressing liquid may be directed generally along the laser energy path. A remote visualization device may be used to view along the laser energy path. The laser energy path can be shrouded with a circumferentially extending suction manifold so that liquid can be suctioned from the target site and away from the laser energy path. In some examples, the suction manifold is an axially extendable and contractible suction manifold.

### RELATED APPLICATION INFORMATION

The present application is a divisional of co-pending patent application Ser. No. 11/671,071, filed 5 Feb. 2007; which claims the benefit of provisional Patent Application No. 60/765,879, filed 7 Feb. 2006.

The present application is related to the following: U.S. Pat. No. 7,063,694 issued 20 Jun. 2006; U.S. Pat. No. 6,986,764, issued 17 Jan. 2006; U.S. Pat. No. 6,554,824 issued 29 Apr. 2003; and U.S. patent application Ser. No. 10/279,087, filed 23 Oct. 2002 (abandoned).

### BACKGROUND OF THE INVENTION

1. Field of the Invention

The present invention relates generally to laser treatment of tissue, and more particularly to the laparoscopic resection, vaporization and coagulation of tissue, such as prostate, kidney and liver tissue, in a hemostatic and photoselective fashion.

2. Description of Related Art

A commonly employed procedure for removal of tissue in the treatment of various medical conditions involves the use of a laparoscopic laser device. Laparoscopic surgery typically involves insufflating the bodily cavity, typically the abdominal cavity, with a gas such as carbon dioxide. Lasers having different wavelengths, power outputs, and pulsing schemes are chosen according to the particular procedure, that is the tissue being treated, the environment and what is to be accomplished. For example, in urology a laser having a wavelength of 532 nm may be chosen for treatment of benign prostatic hyperlasia (BPH) while a laser having a wavelength of 2100 nm is often chosen for treatment of stones in the urinary tract.

### SUMMARY OF THE INVENTION

The goal of laparoscopic laser procedures is to hemostatically ablate or incise tissue by means of vaporization. Hemostasis is achieved when residual heat induces a zone of coagulation in the tissue. Photoselective vaporization of tissue, such as tissue subject of removal for during a laparoscopic procedure, is based upon applying a high intensity radiation to tissue using a radiation that is highly absorptive in the tissue, while preferably being absorbed only to a negligible degree by water or other irrigant during the operation, at power densities such that the majority of the energy is converted to vaporization of the tissue with a small volume of residual coagulation of adjacent tissue. Embodiments are described in which wavelengths absorbed by the smoke suppressing irrigant can be used, by directing the liquid in a pattern around the target without requiring the laser radiation to pass through a significant amount of the liquid.

A drawback associated with using lasers in laparoscopic surgery is that the vapor, mist, gases and smoke, hereinafter commonly collectively referred to as smoke, typically produced by the laser light acting upon the target tissue can make it very difficult for the physician to see what is actually happening at the target tissue, and interfere with the radiation being applied for vaporization of the tissue. The smoke can prevent the physician from properly vaporizing the target tissue. One of the primary aspects of the invention is the recognition that if one were to irrigate the target tissue, such as along the laser light path from the tip of the instrument to the target tissue, the irrigating liquid would capture the smoke and aid visualization of the target site. By the appropriate choice of the irrigating liquid and/or the wavelength of the laser light, the amount of the laser light energy absorbed by the irrigating liquid can be substantially reduced or effectively eliminated. This provides the dual advantages of allowing more energy to reach the target tissue and reducing heating of the irrigating liquid. The latter is important because the irrigating liquid can help cool the surrounding tissue to protect the surrounding tissue from preventable damage. Also, substantially reducing or effectively eliminating the absorption of laser light energy by the irrigating liquid helps to prevent the irrigating liquid from vaporizing, which would itself interfere with the view of the target tissue and the ability of the irrigating liquid to effectively suppress any smoke created by the laser light acting on the target tissue.

It has been recognized that as more and more laser energy is consumed by vaporization of the tissue, the amount of laser energy leading to residual tissue coagulation gets smaller, i.e. the amount of residual coagulation drops, and the side effects attendant to the residual injury caused by the surgery drop dramatically. Thus, the extent of the zone of thermal damage characterized by tissue coagulation left after the procedure gets smaller with increasing volumetric power density, while the rate of vaporization increases. Substantial and surprising improvement in results is achieved. It has been recognized that increasing the volumetric power density absorbed in the tissue to be ablated has the result of decreasing the extent of residual injury of the surrounding tissue. This recognition leads to the use of higher power laser systems, with greater levels of irradiance at the treatment area on the tissue, while achieving the lower levels of adverse side effects and a quicker operation times.

According to an embodiment described herein, a method includes delivering laser radiation to the treatment area on the tissue, via an optical fiber for example, wherein the laser radiation has a wavelength and irradiance in the treatment area on the surface of the tissue sufficient to cause vaporization of a substantially greater volume of tissue than a volume of residual coagulated tissue caused by the laser radiation. In one embodiment, the laser radiation is generated using a neodymium doped solid-state laser, including optics producing a second or higher harmonic output with greater than 60 watts average output power, and for example 100 watts average output power, or more. The laser radiation is coupled into an optical fiber adapted to direct laser radiation from the fiber to the treatment area on the surface of the tissue.

In other embodiments, the delivered laser radiation has a wavelength in a range of about 300 nm to about 700 nm, with smoke suppressing irrigant comprising water, and has an average irradiance in the treatment area greater than about 5 kilowatts/cm², and a spot size of at least 0.05 mm². More preferably, the irradiance is greater than about 10 kilowatts/cm², and even more preferably greater than about 30 kilowatts/cm². Other wavelengths suitable for particular operations can be used, including for example wavelengths in the infrared regions, including about 1 to 10 microns. A first aspect of the present invention is directed to a laparoscopic laser device, for use with an insufflated bodily cavity. The device includes an elongate body having a proximal end and a distal end, the body being adapted for insertion into an insufflated bodily cavity. A laser energy delivery element is coupleable to a source of tissue-vaporization-capable laser energy and is at the distal end of the elongate body. The laser energy delivery element is capable of delivering laser energy along a laser energy path, the laser energy path extending away from the laser energy delivery element. A smoke-suppressing liquid pathway extends along the elongate body to an exit opening at the distal end of the elongate body. The liquid pathway is coupleable to a source of a smoke-suppressing liquid. The liquid pathway at the exit opening is configured to direct the smoke-suppressing liquid generally along the laser energy path.

In some embodiments invention may comprise a remote visualization device having an image receiving portion at the distal end of the elongate body to permit a user to view a region generally along the laser energy path. The elongate body may have a deflectable distal end, the distal end placeable in at least two orientations. The invention may also have an illuminating element having a light discharge portion at the distal end of the elongate body.

A second aspect of the invention is directed to a method for treating tissue at a target site within a patient. A bodily cavity of a patient is insufflated. A distal portion of an elongate body of a laparoscopic laser device is placed at a target site within the insufflated bodily cavity. Tissue-vaporization-capable laser energy is directed along a laser energy path from the distal portion of the body towards the target site thereby vaporizing target site tissue. Smoke created by vaporizing tissue at the target site is suppressed by flowing a liquid generally along the laser energy path.

In some embodiments the laser energy directing step and the aqueous fluid flowing step are carried out so that the laser energy is effectively unabsorbed by the aqueous fluid. The target site may be selectively illuminated and remotely viewed.

A third aspect of the invention is directed to a method for photoselective vaporization of tissue. A bodily cavity of a patient, containing target tissue, is insufflated. Laser radiation and a flow of a transparent liquid irrigant are delivered generally along the laser energy path, to a treatment area on a surface of target tissue. The laser radiation causes vaporization of a volume of tissue greater than a volume of residual coagulation of tissue. The laser radiation has irradiance in the treatment area greater than 10 kiloWatts/cm² in a spot size at least 0.05 mm².

Other aspects and advantages of the present invention can be seen on review the figures, the detailed description, and the claims which follow.

### BRIEF DESCRIPTION OF THE DRAWINGS

FIG. 1 is a simplified overall view of a laparoscopic laser system made according to the invention;

FIG. 2 is a graph of wavelength versus absorption coefficient for water and oxyhemoglobin;

FIG. 3 is a simplified view showing both irrigating liquid and laser light extending along a laser energy path from the distal end of the body of the device of FIG. 1 to a target tissue site;

FIG. 4 is an enlarged view of the distal end of the body of FIG. 3;

FIG. 5 is a simplified overall view of an alternative embodiment of the laparoscopic laser device of FIG. 1;

FIG. 6 is a view similar to that of FIG. 4 of an alternative embodiment of the invention in which the irrigation pathway is generally coaxial with and surrounds the exit of a laser energy delivery element;

FIG. 7 is a view similar set of FIG. 3 of an alternative embodiment using a side firing laser energy delivery element;

FIG. 7A is a simplified partial side view of a further alternative embodiment of the laparoscopic laser device of FIG. 1;

FIG. 8 is a simplified diagram of a diode pumped, solid-state laser system producing over 100 Watts frequency converted output power;

FIG. 9 is a graph of absorption efficiency versus wavelength for pump energy sources in an Nd:YAG gain medium;

FIG. 10 illustrates one end of a gain medium in a system such as described with reference to FIG. 8;

FIG. 11 is a schematic illustration of the distribution of pump energy at one end of the gain medium for a system such as described with reference to FIG. 8;

FIG. 12 illustrates in intensity profile on at least one dimension of the pump energy delivered to one end of the gain medium for a system such as described with reference to FIG. 8; and

FIG. 13 is a heuristic diagram illustrating operational characteristics of the system of FIG. 8.

### DETAILED DESCRIPTION

The following description of the invention will typically be with reference to specific structural embodiments and methods. It is to be understood that there is no intention to limit the invention to the specifically disclosed embodiments and methods but that the invention may be practiced using other features, elements, methods and embodiments.

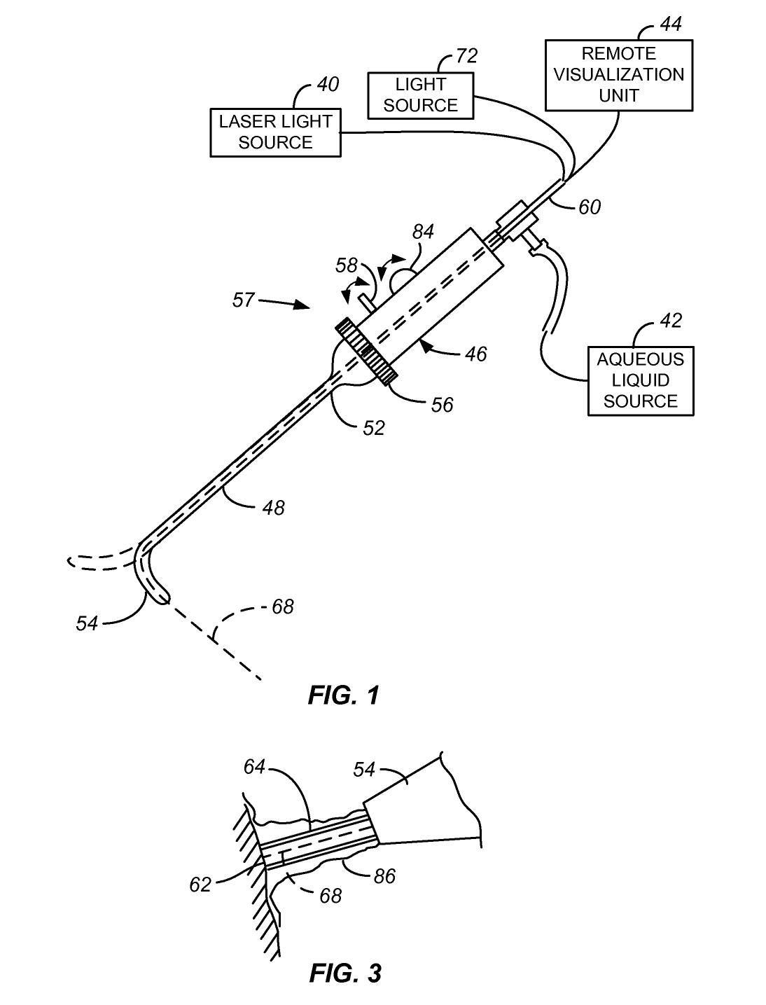

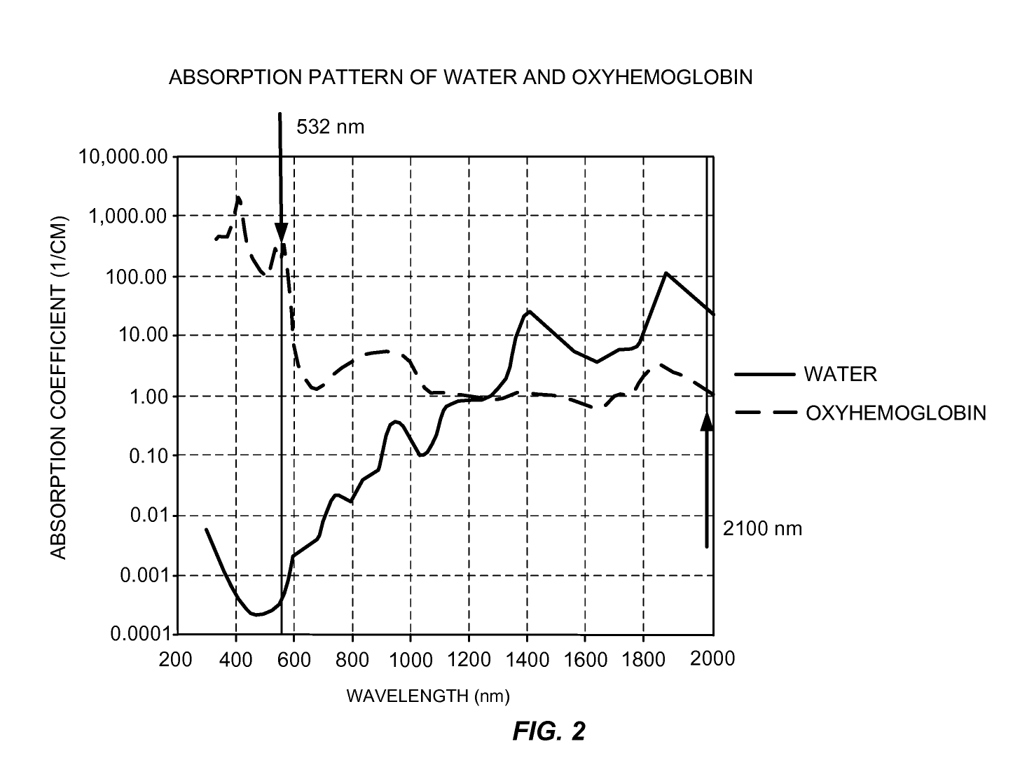

FIG. 1 illustrates a laparoscopic laser system including a laparoscopic laser device 38 coupled to a laser light source 40, also called a laser energy source 40, an aqueous liquid source 42 and a remote visualization unit 44. The laser energy source is chosen so that the laser energy is only minimally absorbed by the irrigating liquid used, typically an aqueous liquid. FIG. 2 is a graph illustrating the absorption pattern of water and oxyhemoglobin. The absorption coefficient of water for laser wavelengths of 400-600 nm is extremely low, with the absorption coefficient of lasers having a wavelength of 532 nm being plotted on the graph. At the same time laser wavelengths of 400-600 nm, and in particular of 532 nm, are highly selectively absorbed by oxyhemoglobin in tissue allowing for efficient photoselective tissue heating. While it is preferred that when an aqueous irrigating liquid is used, the laser wavelength be between 400 and 600 nm, in some situations laser wavelengths between about 400 to 800 nm may be effective when, for example, an aqueous irrigating liquid is used. Irrigating liquids other than an aqueous liquid may be used in appropriate cases. Although wavelengths in the blue light range of about 400-425 nm are especially attractive, at present practical difficulties restrict their widespread use.

The laparoscopic laser device 38 of FIG. 1 includes a handle 46 from which an elongate body 48 extends. The elongate body has a proximal end 52 connected to the handle and a deflectable distal end 54. The deflectable distal end 54 is placeable in at least two orientations, and typically a range of orientations. The distal end 54 may be bendable or rotatable, and typically is both bendable and rotatable. The deflectable distal end 54 of the body 48 can be rotated by manipulating a wheel 56 of a steering assembly 57 at the distal end of the handle 46; this eliminates the need to rotate the entire handle when it is desired to rotate the distal end 54 of the body 48. The distal end 54 of the body 48 can also be curved or bent or otherwise deflected to point in different directions by manipulating a deflection device 58 of the steering assembly 57, also mounted to the handle 46. Catheters having rotatable and deflectable tips are generally known; see, for example, U.S. Pat. Nos. 6,571,131; 5,545,200; 6,572,643: and 6,238,430.

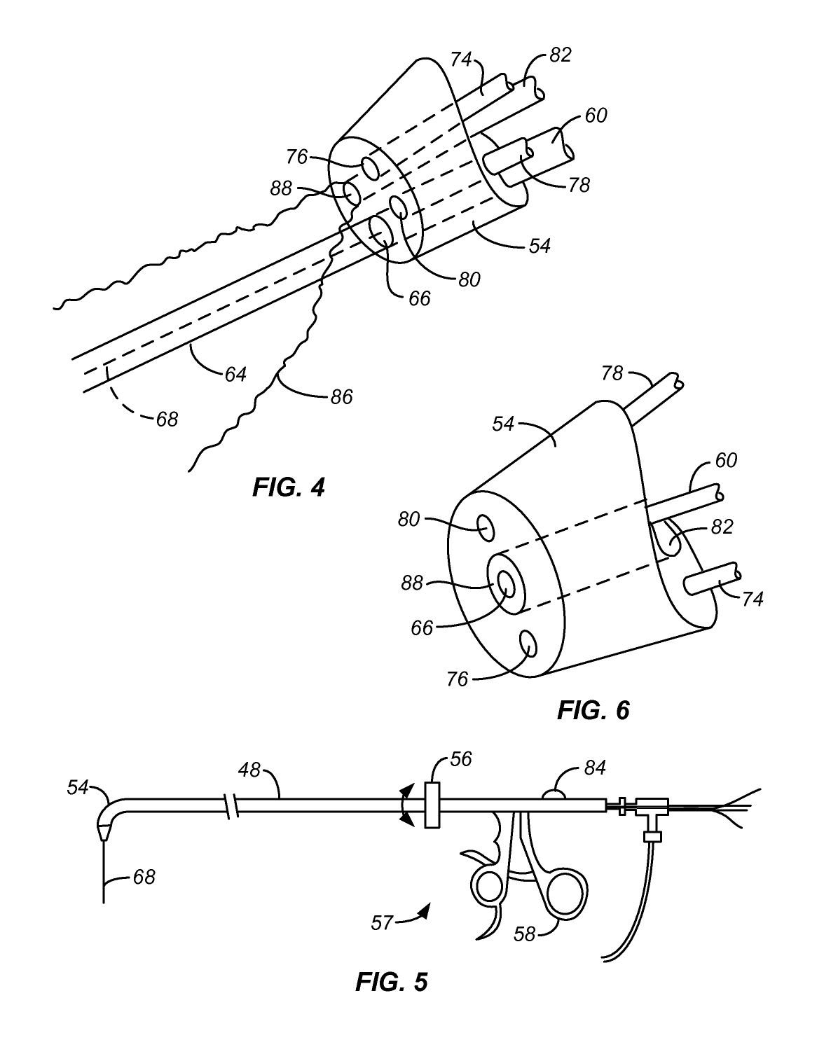

A fiber optic laser energy delivery element 60 is connected to the laser energy source 40 at the handle 46 and delivers laser energy to a target tissue site 62; see FIG. 3. The laser light 64, see FIG. 4, passes from an exit 66 of the laser energy delivery element 60 along a laser energy path 68. For rapid procedures, according to the present invention, the spot size at the target tissue should be large enough that the operator can remove tissue at a reasonable rate, and see the results of a single pass of the spot over a region of tissue. If the spot size is too small, the rate of the operation can be too slow for a given energy density. Also, if the spot size is too big, then some of the more precision procedures will be difficult to control precisely. A preferred spot size for a precision process is less than about 1 mm², and more particularly between about 0.8 mm² and about 0.05 mm². Other apparatus may be used for delivery of the beam with the desired spot size, including embodiments without diverging beams, and embodiments with converging beams.

Selective illumination of the target site may be provided by an illumination element 70 including a light source 72, see FIG. 1, connected to an illumination light guide 74, see FIG. 4, passing through the laparoscopic laser device 38.

The illumination light guide 74 typically includes a light cable, extending from the light source 72, and glass fibers, connected to the light cable and extending along the elongate body 48. Illumination light from the light source can, when needed, be directed towards the target tissue site 62 through the tip 76 of the illumination light guide 74. Other types of illumination elements can also be used. For example, a light emitter, such as one or more LEDs, can be mounted at the distal end of the body and selectively connected to an appropriate energy source by wires, extending through the elongate body, and a user-operated switch. Illumination of the target tissue site may also be accomplished using a device separate from the device of FIG. 1.

A remote visualization device 78 has an image receiving portion 80 at the distal end 54 of the body 48 connected to the remote visualization unit 44 by an optical fiber or other appropriate structure. The remote visualization device 78 may be of the type having, for example, an optical lens arrangement or a semiconductor image sensor as the image receiving portion; such remote visualization device 78 would be connected to the remote visualization unit 44 in an appropriate manner.

A lumen through the elongate body defines an irrigation pathway 82 connected to the liquid source 42. The flow the aqueous irrigating liquid 86 is controlled by an irrigation control 84 on the handle. Smoke suppressing liquid 86, such as water, saline solution or other biocompatible material, passes through the liquid exit port 88 at the distal end 54 of the body 48. The irrigation pathway 82 at the exit port 88 is configured to direct the aqueous irrigating liquid 86 along the laser energy path as suggested in FIGS. 3 and 4. This causes the irrigating liquid 86 to suppress smoke caused by the laser energy acting on the target tissue at the target tissue site 62. This permits improved viewing of the target tissue site 62 by the physician using the remote visualization unit 44, which is provided an image by the remote visualization device 78. If desired a suction pathway, not shown, may be provided within or along the elongate body to permit spent irrigation liquid and dislodged tissue fragments to be removed from the target site. Alternatively, a suction instrument separate from the device of FIG. 1, not shown, may be used for this purpose. In some situations may be desired to place the elongate body 48 within the bore of the suction instrument.

The device can be controlled to coordinate the timing of the flow of irrigation, the delivery of radiation and the imaging system, to provide images of the procedure that are as unobstructed as possible. For example, the imaging system can be controlled in an embodiment to take images between sets of pulses of radiation and smoke suppressant, where the sets can include from one to many pulses depending on the pulse rate and the imaging quality desired. For an illustrative example, using laser pulse rates at 10 kHz, the pulse sets could be arranged in sets of about 500 pulses with continuous flow smoke suppressant during the pulse set, followed by one image with the laser and flow off between pulse sets. This could produce for in the neighborhood of 10 to 15 images per second. Of course, these parameters can be empirically determined.

The present invention can be used in various situations involving the laser treatment of tissue. However, invention is particularly suited for the laparoscopic resection, vaporization and coagulation of tissue, such as prostate, kidney and liver tissue, in a hemostatic and photoselective fashion.

In one exemplary use, a laparoscopic partial nephrectomy may be performed by placing the distal portion of the elongate body of the laparoscopic laser device at a target site of the kidney. The laser light, in this example, has a wavelength of 532 nm. The physician can inspect the target site using the remote visualization unit 44, the target site 62 typically being illuminated using the light source 72. Laser energy 64 is then directed at the target site 62 and the aqueous irrigation liquid 86 is directed from the distal end 54 of the body 48. The energy level of the laser light 64 and the flow rate of the irrigation liquid 86 are preferably both controllable. The aqueous liquid 86 not only suppresses smoke created during the lasing procedure but it also helps to cool the surrounding tissue. A suction device is preferably used along with or as a part of the laser device to suction away the irrigating liquid together with smoke and tissue debris. The partial nephrectomy is typically performed by one of two techniques. The laser light can be used to vaporize the targeted renal parenchyma to the desired size and depth by passing the laser light over the entire desired area of resection thereby completely vaporizing the target tissue. Alternatively, a wedge resection procedure may be conducted by using the laser light as a cutting tool to excise the target tissue, which can then be retrieved as a partial nephrectomy specimen. In the event of hemorrhage, the power level of the laser light can be reduced, or the laser light can be defocused, so that the laser light has a hemostatic effect. Other measures for hemostasis are typically not required with the present invention. Similar procedures for treating other types of tissues, such as the prostate, may be used.

As used in this application, effectively unabsorbed means that the laser energy (1) passes through the smoke-suppressing liquid without raising the temperature of the liquid more than for example, 40° C., and (2) has sufficient energy after passing through the liquid to vaporize the target tissue. This depends primarily on the absorption coefficient for the particular wavelength and irrigating liquid.

FIG. 5 illustrates an alternative embodiment of the laparoscopic laser device 38 of FIG. 1. The primary differences relate to the steering assembly 57 in which the deflection device 58 is a pistol grip type of structure. FIG. 6 illustrates the distal end of the body of another alternative embodiment of the device of FIG. 1. In this case the irrigation pathway 82 and the laser energy delivery element 60 are, at the distal end of the body, generally coaxial with the irrigation pathway surrounding the exit 66 of the laser energy delivery element 60 to help ensure flow of the irrigating liquid 86 along and surrounding the laser energy path 68.

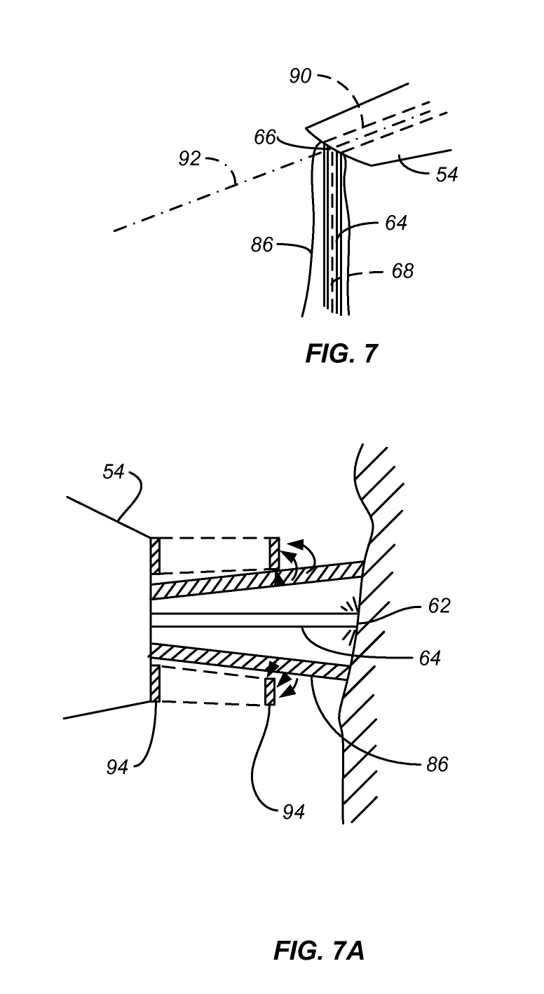

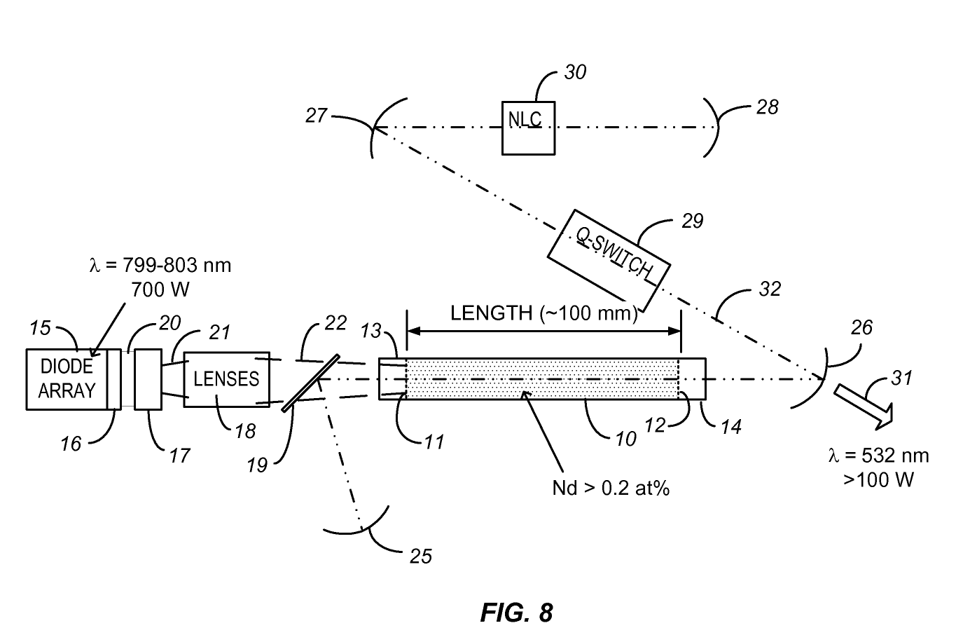

The use of a body with a deflectable distal end helps the user to direct the laser light at the appropriate location at the target tissue site. In some cases it may be desired to use what is called a side firing laser energy delivery element 90. In this case the laser energy path is at an angle, and often perpendicular to, the centerline 92 of the laser energy delivery element 90, typically a fiber-optic element, at the exit 66. This is illustrated in FIG. 7.

In some situations it may be desired to use laser light at wavelengths that are not effectively unabsorbed by aqueous liquids or other physiologically suitable smoke-suppressing irrigation liquids. Rather than directing the irrigation liquid coincident with the laser energy path so that the laser light passes through the liquid prior to contacting the target tissue, the irrigation liquid could be directed to be offset from, for example to the side of, the laser energy path. For example, the irrigation liquid 86 could be directed to one or more sides of the laser energy. Also, the smoke suppressing irrigation liquid 86 could be offset from the laser energy path by being directed in a hollow tube or cone with the laser light 64 passing through the hollow center. See FIG. 7A. The smoke suppressing liquid may be, for example, in the form of a mist, vapor or fine spray. To help prevent the laser light from passing through any substantial amount of the smoke suppressing liquid, one or more suction ports 94 may be provided at the distal end 54 of the body 48 to draw away irrigation liquid, tissue particles and smoke from the target site. Alternatively, suction could be provided through one or more separate suction devices. In one embodiment the suction device could be configured as a circular manifold encircling the target tissue site. Such a circular manifold could be a part of separate suction device or it could be extended from the distal end of the body as indicated in dashed lines in FIG. 7A.

The laser energy source may, in different embodiments, provide laser energy at power levels of at least about 40 W, 60 W and 100 W average output power. The following provide information on laser energy sources capable of producing these types of energy levels, the disclosures of which are incorporated by reference: U.S. patent application Ser. No. 10/371,080 filed 21 Feb. 2003; U.S. Pat. No. 6,986,764 issued 17 Jan. 2006; U.S. Pat. No. 6,554,824 issued 29 Apr. 2003.

FIG. 8 illustrates a high-power laser system comprising a gain medium 10 that includes a doped crystalline host, having a first end 11 and a second end 12. The gain medium 10 in a representative embodiment comprises Nd:YAG having a length of about 100 millimeters and a diameter of about 4.5 millimeters. The gain medium 10 is water cooled in exemplary embodiments, along the sides of the host. Undoped endcap 13 about 10 millimeters long in this example, is bonded on the first end 11 of the gain medium 10, and undoped endcap 14 also about 10 millimeters long in this example, is bonded on the second end 12 of the gain medium 10.

In the high-power end-pumped configuration shown, the undoped endcap 13 can be diffusion bonded but preferably grown on at least the first end 11. In embodiments where significant pump energy reaches the second end of the host 10, another undoped endcap 14 can be diffusion bonded but preferably grown on the second end 12. The output end of the undoped endcap 14 is coated so that it is reflective at the pump energy wavelength, while transmitting at the resonant mode. In this manner, the pump energy that is unabsorbed at the second end 12 is redirected back to the rod to be absorbed. At the very high pump powers possible using the configuration described herein, rod-end lens effects play a very significant role in the stability of the resonator. Strong absorption of the pump energy at the surface of the gain medium can cause significant distortion to the end face and at high-power levels rod fracture. Rod distortion leads to strong spherical aberration of the beam which severely reduces the quality of the beam. By bonding undoped endcaps onto the doped rod ends, the distortion is avoided, because the absorption now takes place in the bulk and not at a surface. Also, the fracture limit is higher and end effects are substantially eliminated.

A source of pump energy in the illustrated embodiment comprises a diode array 15. A representative embodiment employs a seven bar stack of diode lasers, with each bar producing 100 Watts for 700 Watts total pump energy, centered on 801 nanometers. The wavelength of the bars changes plus or minus 1.5 nanometers in normal operating conditions providing pump energy within a range of about 799 to about 803 nanometers.

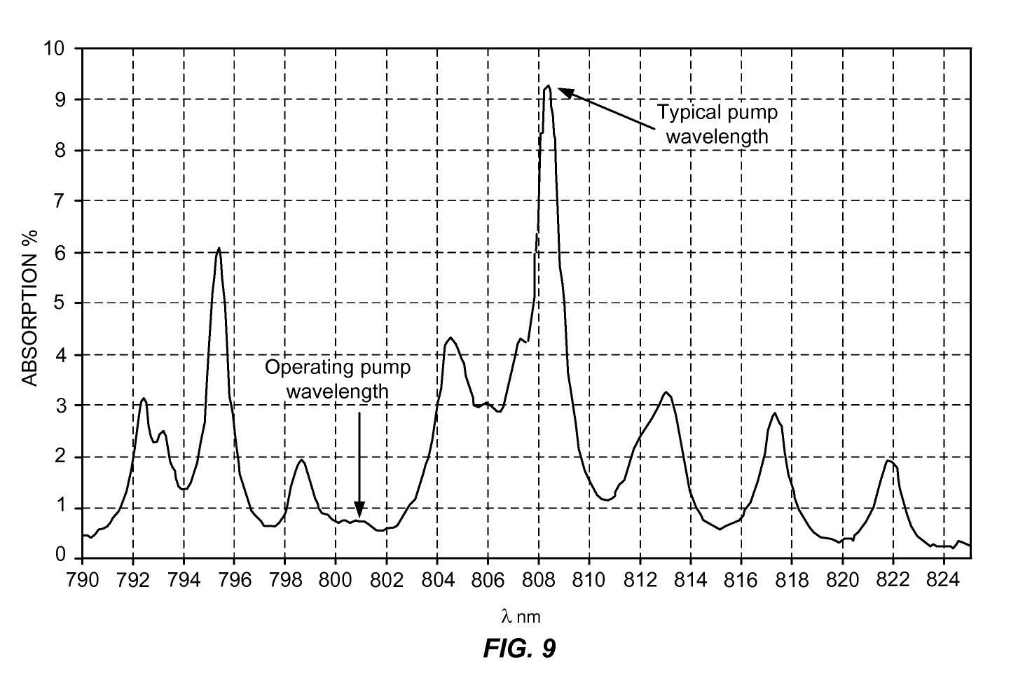

FIG. 9 shows the absorption efficiency versus pump energy wavelength over practical range of wavelengths, for Nd:YAG. As shown, a maximum in the range occurs at about 808 nanometers. The pump energy range of 799 to 803 lies substantially off the peak at 808, at a level that is less that 20 percent of the maximum absorption. For 801, plus or minus 1.5 nanometers, the absorption is less than about 10% of the maximum absorption at the peak near 808 nanometers. Other pump energy ranges are suitable as well, including wavelengths near 825 nanometers or beyond the illustrated range. One specific advantage of pumping at wavelength with absorption efficiencies that are substantially off peak is a tolerance to wavelength shifts. When pumping at 801 nanometers in the Nd:YAG in the described embodiment, wavelength shifts of plus or minus 1.5 nanometers have essentially no effect on the laser output.

Pump energy is delivered through optics, including a fast axis collimation lens 16, a polarization multiplexer which acts as a beam interleaver, brightness doubler 17, and a set of lenses 18 arranged as a telescope to focus the pump energy near the first end 11 of the gain medium 10. The pump energy is delivered at the output of the fast access collimation lenses 16 on a path 20 to the beam interleaver, brightness doubler 17. The pump energy is concentrated to one half its width at the output of the beam interleaver, brightness doubler 17 on path 21 and is delivered through the lenses 18 on path 22 to a focal point at or near the first end 11 of the gain medium 10.

In embodiments of the invention, the fast axis collimation lens 16 can be deliberately defocused slightly to facilitate homogenization of the pump beam at the focal point in the gain medium 10. The beam interleaver, brightness doubler 17 reduces the width of the pump energy output by one half, facilitating focusing of the pump energy into a relatively small diameter rod shaped gain medium 10, with a longer working distance. The lenses 18 can be varied to adjust the spot size at an image plane in the gain medium 10 over a range of operating parameters as suits a particular implementation. For example, the spot size at the focal point can be varied over range about 10 percent to about 90 percent of the diameter of the rod shaped gain medium 10.

The pump energy passes through a beam splitter 19 that is used to turn the resonating energy to the optics defining resonant cavity. The system includes optical elements including concave mirror 25, that is highly reflective at the resonating energy of 1064 nanometers, beam splitter 19, which is reflective at 1064 nanometers and transmissive at the wavelength of the pump energy source around 801 nanometers, concave mirror 26 that is highly reflective at 1064 nanometers and transmissive at an output wavelength of 532 nanometers, concave mirror 27 that is highly reflective at both 1064 and 532 nanometers, and concave mirror 28 which is highly reflective at both 1064 and 532 nanometers. The optical elements 25, 19, 26, 27, 28 define a resonant path 32 which is essentially Z-shaped, with a tail between then beam splitter 19 and the highly reflective concave mirror 25.

In the illustrated embodiment, Q-switch 29 is placed in the resonant cavity between the mirrors 26 and 27. Also, a nonlinear crystal 30, such as LBO, is placed between the mirrors 27 and 28. The Z-shaped resonant cavity can be configured as discussed in U.S. Pat. No. 5,025,446 by Kuizenga, imaging the resonant mode at one end of the gain medium 10 at the nonlinear crystal 30. The configuration described is stable and highly efficient for frequency conversion. The configuration shown in FIG. 1 produces a frequency converted output (wavelength 532 nanometers in illustrated embodiment) of greater than 100 Watts on line 31.

The pump spot size at the image plane near the first end 11 of the gain medium 10 affects in the mode quality of the laser system, controls the gain, and the strength of the thermal lensing.

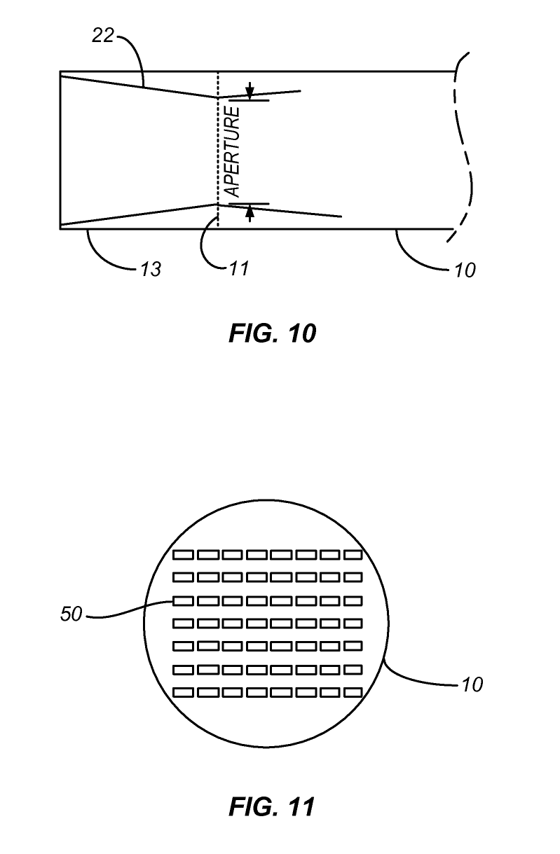

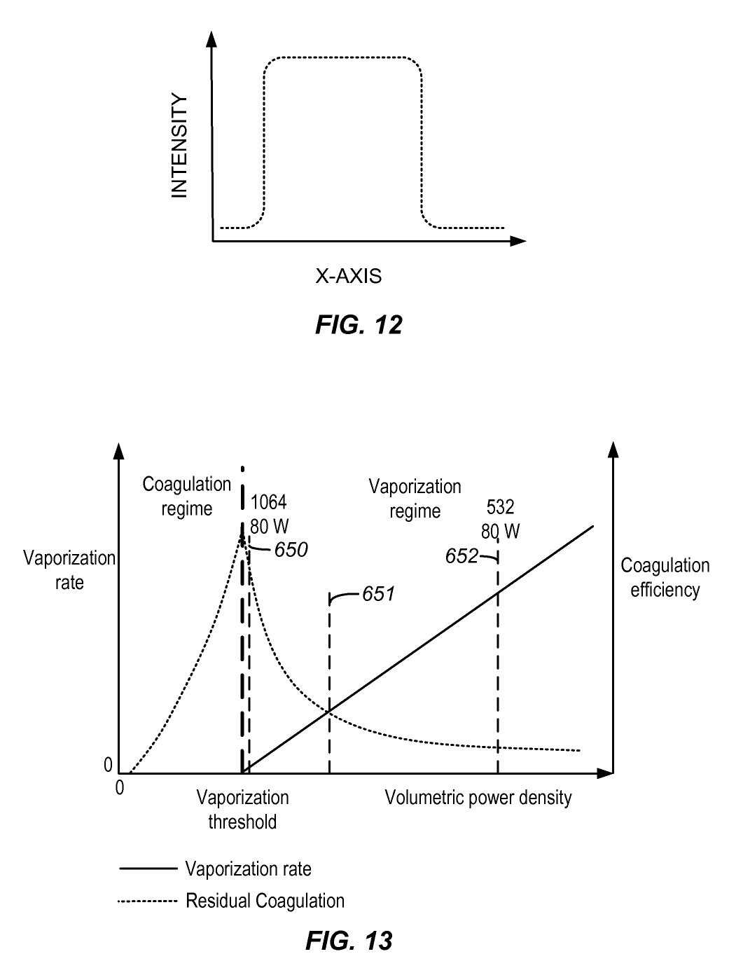

FIGS. 10 and 11 illustrate features of the pump spot size at the focal point. FIG. 2 shows the gain medium 10, and the undoped endcap 13 on the first end 11 of the gain medium 10. The pump energy is focused on path 22 to the focal point near the first end 11. This establishes an aperture near the first end for the resonant mode in the cavity. The gain is inversely proportional to the area and divergence of the pump beam at the focal point near the first end 11 of the gain medium 10 at the doped/undoped interface of the rod. The smaller the spot size, the high the gain for a given rod. The thermal lens is also inversely proportional to the pump spot size at the image plane. As the pump spot gets smaller, the thermal lens increases. Also, the distribution of light across the pump spot has a strong effect on the thermal lens. FIG. 11 illustrates the distribution light from the pump energy source at the first end 11 on the rod, which results from imaging the output of the laser diode source on the first end 11 of the rod. As illustrated in FIG. 11, there are seven rows of diode laser outputs, such as row 50. The result is a substantially uniform intensity profile, as illustrated in FIG. 12 along the horizontal dimension in the FIG. 12, which lies on an axis that is parallel to the row 50 of laser diode spots. The rows are separated by a small distance in the vertical dimension in an embodiment where the fast axis collimation lenses 16 are focused. By slightly defocusing the fast axis collimation lenses 16, the distribution of energy can be made more uniform in the second, vertical dimension. The system is designed therefore to homogenize and flatten the pump profile to reduce the thermal lensing.

Also, the spot size at the image plane affects transverse modes of the laser. The transverse modes of the laser are controlled by the pump spot size and distribution of energy within about the first 30 percent of the rod length in which a most of the pump energy is absorbed. As the spot size at the image plane is reduced, the mode quality improves. The optical elements 25, 19, 26, 27, 28 defining the resonant cavity are configured to mode match with the aperture defined by the pump energy spot size at the focal point.

The doping concentration in the gain medium 10 is chosen based on the mode quality and output power required. The doping level is relatively low to allow distribution of the thermal load along the optical axis of the gain medium 10 (e.g., 1/e absorption length of more than 50 millimeters in a rod less than 10 millimeters in diameter), thereby reducing the thermal stresses induced at the input to the gain medium. In an embodiment described, the doping concentration is about 0.27 atomic percent for the rod shown in FIG. 8, that is about 100 millimeters long between the first end 11 and the second end 12, and pumped substantially off-peak at about 801 nanometers where the absorption efficiency is less than 10 percent of the maximum absorption efficiency at the peak near 808 nanometers for Nd:YAG. The 1/e absorption length for this embodiment is about 66 millimeters, more than half the length of the 100 millimeters rod.

Ranges of doping concentrations for embodiments of the invention comprising an Nd:YAG rod can fall within about 0.05 and about 0.5 atomic percent, and more preferably in a range between about 0.2 and 0.4 atomic percent for readily and consistently manufacturable commercial applications. The pump energy wavelength, doping concentration and the length of the rod are adapted in a preferred embodiment, so that the absorption length is over one third the rod length, and more than 90 percent of the pump energy is absorbed within two passes along the length of the rod, as the unabsorbed pump energy which reaches the second end 12 of the rod is reflected back towards the first end 11. The amount of unabsorbed pump energy that reaches the first end 11 is very low, and has insubstantial effects on the characteristics of the pump energy at the focal point.

By establishing a suitable combination of parameters including the length for the gain medium, the doping concentration, the pump energy profile at the image plane, and the pump energy wavelength, output powers greater than 100 Watts of frequency converted output at 532 nanometers are readily generated with an Nd:YAG rod about 100 millimeters long and about 4.5 millimeters in diameter with reasonably high quality beam. The technology is scalable to configurations supporting pump energy in the kilowatt range for hundreds of Watts of output power in the primary and harmonic wavelengths for the laser.

Beam quality can be characterized by the parameter M². The higher M², the lower the beam quality, and the more difficult it is to focus of the beam on a small spot and to couple the beam into small numerical aperture delivery devices such as fiber optics. M² of less than 30 is readily achieved using the technology described herein, allowing coupling into fiber optics on the order 100 microns and up in diameter, which provides a beam with low divergence suitable for many high-power applications of laser light, including medical applications.

The technology described herein is adaptable to other configurations of the resonant cavity, with or without frequency conversion and with or without Q-switching, and adaptable to other gain media and pump energy sources within the parameters described herein.

For rapid procedures, according to the present invention, the spot size should be large enough that the operator can remove tissue at a reasonable rate, and see the results of a single pass of the spot over a region of tissue. If the spot size is too small, the rate of the operation is too slow. Also, if the spot size is too big, then the procedure is difficult to control precisely. A preferred spot size is less than about 1 mm², and more particularly between about 0.8 mm² and about 0.05 mm². Other apparatus may be used for delivery of the beam with the desired spot size, including embodiments without diverging beams, and embodiments with converging beams.

FIG. 13 shows, heuristically, how vaporization rate and coagulation rate depend on the volumetric power density. The vaporization rate (in mm/s) is defined as tissue depth that is vaporized per time interval. The coagulation rate (in mm/s) is defined as the depth of residual coagulated tissue that remains after a certain time of vaporization.

Below a certain volumetric power density, referred to as a “vaporization threshold” in FIG. 13, no tissue gets vaporized. All laser energy stays inside the tissue. Tissue coagulation occurs where the tissue temperature rises above approximately 60° C. As the volumetric power density is increased a bigger and bigger tissue volume gets coagulated.

At the vaporization threshold, vaporization starts. Above the vaporization threshold the vaporization rate can be considered to increase linearly with the volumetric power density for the purpose of understanding the present invention, and as described by a steady state model for continuous wave laser tissue ablation, known by those familiar with the art of laser-tissue interaction.

As more and more laser energy is consumed by vaporization of the tissue, the amount of laser energy leading to residual tissue coagulation gets smaller, i.e. the amount of residual coagulation drops. Thus, extent of the zone of thermal damage characterized by tissue coagulation left after the procedure gets smaller with increasing volumetric power density, while the rate of vaporization increases. Substantial and surprising improvement in results is achieved.

Publications about visual laser ablation of the prostate (VLAP) that is performed with an Nd:YAG laser at 1064 nm have shown that this type of laser is not able to vaporize a significant amount of tissue. Histology studies have shown that the 1064 nm laser induces deep coagulation in the tissue that results in edema and delayed tissue sloughing. This effect was described by Kuntzman, et al., High-power potassium titanyl phosphate laser vaporization prostatectomy. Mayo Clin Proc 1998:73:798-801.

As the laser power is further increased to 80 W, and the side firing probe is placed less than 1 mm from the tissue for a small spot size, the ablation rate further increases and the coagulation rate further drops, so that the procedure lies heuristically at point 652 in FIG. 13.

An 80 Watt laser at green wavelengths can be used to easily reach irradiance levels that vaporize substantially more tissue than is left as residual coagulation after the procedure. More precisely, the vaporization rate is substantially higher than the coagulation rate as given by the definition above, using high irradiance levels that are easily achieved with higher power lasers. Because of higher vascularization in the uterus, the optical penetration depth is lower than in prostatic tissue, and therefore the volumetric power density at the vaporization threshold can be easily reached with lower average power lasers, including for example a 40 W average output power laser. Other laser systems generating wavelengths in the infrared including Holmium based lasers and CO₂ based lasers could be utilized.

The above descriptions may have used terms such as above, below, top, bottom, over, under, et cetera. These terms are used to aid understanding of the invention are not used in a limiting sense.

While the invention has been particularly shown and described with reference to preferred embodiments thereof, it will be understood by those skilled in the art, that various changes in form and details may be made therein without departing from the spirit and scope of the invention, as defined by the appended claims.

Any and all patents, patent applications and printed publications referred to above are incorporated by reference.

1. A laparoscopic laser device, for use with an insufflated bodily cavity, comprising:
an elongate body having a proximal end and a distal end, the body adapted for insertion into an insufflated bodily cavity;
a laser energy delivery element, coupleable to a source of tissue-vaporization-capable laser energy, at the distal end of the elongate body, the laser energy delivery element capable of delivering laser energy along a laser energy path, the laser energy path extending away from the laser energy delivery element;
a smoke-suppressing liquid pathway extending along the elongate body to an exit opening at the distal end of the elongate body, the liquid pathway coupleable to a source of a smoke-suppressing liquid;
the liquid pathway at the exit opening configured to direct the smoke-suppressing liquid generally along the laser energy path; and
a vacuum port at the distal end of the body circumferentially surrounding the laser energy path.

2. The device according to claim 1, wherein the liquid path is configured to direct the smoke suppressing liquid to surround the laser energy path.

3. The device according to claim 1, wherein the vacuum port is an axially extendable and retractable vacuum port manifold placeable at different positions along the laser energy path.

4. A method for treating tissue at a target site within a patient comprising:
insufflating a bodily cavity of a patient;
placing a distal portion of an elongate body of a laparoscopic laser device at a target site within the insufflated bodily cavity;
directing tissue-vaporization-capable laser energy along a laser energy path from the distal portion of the body towards the target site thereby vaporizing target site tissue; suppressing smoke created by vaporizing tissue at the target site by flowing a liquid generally along the laser energy path;
surrounding the laser energy path with a circumferentially extending suction manifold; and
suctioning liquid from the target site and away from the laser energy path through the suction manifold.

5. The method according to claim 4, wherein the surrounding step comprises surrounding the laser energy path with a circular suction manifold.

6. The method according to claim 4, wherein:
the surrounding step comprises surrounding the laser energy path with an axially extendable and retractable suction manifold; and
placing the suction manifold at different positions along the laser energy path.

7. The method according to claim 4, wherein the insufflating step is carried out on an abdominal cavity of a patient.

8. The method according to claim 4, wherein the laser energy directing step comprises directing laser energy having a wavelength of about 400 to 800 nm and the smoke suppressing step is carried out using an aqueous liquid as the liquid.

9. The method according to claim 4, wherein the laser energy directing step comprises directing laser energy having a wavelength of about 532 nm.

10. The method according to claim 4, wherein the laser energy directing step comprises directing laser energy having an average output power of the least 40 W.

11. The method according to claim 4, further comprising remotely viewing the target site.

12. The method according to claim 11, further comprising facilitating the remotely viewing step by selectively illuminating the target site with light from an illuminating element having a light discharge portion at the distal end of the elongate body.

13. The method according to claim 4, wherein the laser energy directing step further comprises remotely deflecting the distal portion of the elongate body.

14. The method according to claim 4, wherein the laser energy directing step is carried out for a least one of resection, vaporization and coagulation of tissue at the target site in a hemostatic and photoselective fashion.

15. The method according to claim 4, wherein the placing step is carried out at a target site of a kidney.

16. The method according to claim 4, wherein the laser energy directing step further comprises remotely deflecting the distal portion of the elongate body.

17. The method according to claim 4, wherein the smoke suppressing step is carried out so that the laser energy is effectively unabsorbed by the liquid so that the laser energy remains tissue-vaporization-capable.

18. The method according to claim 4, wherein the liquid flowing step is carried out by flowing the liquid generally along but offset from the laser energy path.

19. The method according to claim 4, wherein the liquid flowing step is carried out by flowing the liquid generally along and coincident with the laser energy path so that the laser energy passes through the liquid.

20. A method for treating tissue at a target site within a patient comprising:
insufflating a bodily cavity of a patient;
placing a distal portion of an elongate body of a laparoscopic laser device at a target site within the insufflated bodily cavity;
remotely viewing the target site;
facilitating the remotely viewing step by selectively illuminating the target site;
directing tissue-vaporization-capable laser energy, having a wavelength of 400 to 600 nm, along a laser energy path from the distal portion of the elongate body towards the target site to vaporize tissue at the target site;
the laser energy directing step further comprising remotely deflecting the distal portion of the elongate body;
enhancing the remotely viewing step by:
suppressing smoke at the target site created during the laser energy directing step by flowing an aqueous liquid generally along the laser energy path with the laser energy being effectively unabsorbed by the aqueous liquid and remaining tissue-vaporization-capable;
surrounding the laser energy path with a circumferentially extending suction manifold; and
suctioning liquid from the target site and away from the laser energy path through the suction manifold.

21. The method according to claim 20, wherein the surrounding step comprises surrounding the laser energy path with an axially extendable and contractible suction manifold.

22. A method for performing a partial nephrectomy at a target site of a kidney within a patient comprising:
insufflating a bodily cavity of a patient, the bodily cavity containing the patient's kidney;
placing a distal portion of an elongate body of a laparoscopic laser device at a kidney target site;
remotely viewing the target site;
facilitating the remotely viewing step by selectively illuminating the target site; directing tissue-vaporization-capable laser energy, having a wavelength of 400 to 600 nm, along a laser energy path from the distal portion of the elongate body to target tissue at the target site thereby vaporizing kidney target tissue;
the laser energy directing step further comprising a remotely deflecting the distal portion of the elongate body;
enhancing the remotely viewing step by:
suppressing smoke at the target site created during the laser energy directing step by flowing an aqueous liquid generally along the laser energy path with the laser energy being effectively unabsorbed by the aqueous liquid and remaining kidney-tissue-vaporization-capable;
surrounding the laser energy path with a circumferentially extending suction manifold; and
suctioning liquid from the target site and away from the laser energy path through the suction manifold.

23. The method according to claim 22, wherein the surrounding step comprises surrounding the laser energy path with an axially extendable and contractible suction manifold.
